(19) United States
(12) Patent Application Publication
(10) Pub. No.: US 2003/0220675 A1
(43) Pub. Date: Nov. 27, 2003

(54) Method of cooling the central nervous system

(76) Inventor: C. Gene Coin, Long Key, FL (US)

Correspondence Address: Robert M. Downey, ROBERT M. DOWNEY, P.A., Suite 350, 150 E. Palmetto Park Road, Boca Raton, FL, 33432, US

(21) Appl. No.: 10/442,438

(22) Filed: May 21, 2003

Related U.S. Application Data
(60) Provisional application No. 60/382,416, filed on May 21, 2002.

(51) Int. Cl. A61F 7/00

(52) U.S. Cl. 607/105

(57) ABSTRACT

Cerebrospinal fluid is drawn from the subarachnoid space of the spinal canal, using a modified spinal needle, and is passed through a flexible tube to a reservoir. The spinal fluid is cooled, either during passage through the flexible tube, or in the reservoir, and is then returned to, the subarachnoid space through the same tube, or a separate return tube connected to the reservoir. The cooled cerebrospinal fluid circulates back up over the surface of the brain where it is then absorbed into the veins near the vertex of the brain.

### BACKGROUND OF THE INVENTION

1. Field of the Invention

The present invention is directed to a method of cooling the central nervous system and, more particularly, to a method of cooling the cerebrospinal fluid generated in the ventricular system of the brain for protecting the central nervous system during cardiac arrest and other conditions where the central nervous system is at risk.

2. Discussion of the Related Art

In many instances, cardiac arrest can be reversed and the heart can be restarted with the use of electric shock. However, doctors have not figured out how to stop the brain damage that begins minutes after blood flow stops during cardiac arrest. Recent studies suggest that cooling the body just a few degrees appears to prevent brain damage in patients who survive cardiac arrest. Some studies have found that patients were five times more likely to recover from cardiac arrest when under mild hypothermia. It is believed that cooling works by reducing the brain's need for oxygen while suppressing the chemical processes that kill brain cells. Cooling the body, and particularly the brain, may also be useful for stroke, brain injuries and spinal cord injury.

Presently, cooling the body of cardiac arrest victims has been achieved by use of cold air and ice packs. In light of the promising results, a more efficient means to cool the central nervous system is urgently needed.

The present invention provides an efficient means of rapidly cooling the central nervous system under controlled conditions.

### SUMMARY OF THE INVENTION

Cerebrospinal fluid is drawn from the subarachnoid space of the spinal canal, using a modified spinal needle, that is passed through a flexible tube to a reservoir. The spinal fluid is cooled, either during passage through the flexible tube, or in the reservoir, and is then returned to the subarachnoid space through the same tube, or a return tube connected to the reservoir. The cooled cerebrospinal fluid circulates back up over the surface of the brain where it is then absorbed into the veins near the vertex of the brain.

### BRIEF DESCRIPTION OF THE DRAWINGS

For a fuller understanding of the nature of the present invention, reference should be made to the following detailed description taken in conjunction with the accompanying drawings in which:

FIG. 1 is a schematic diagram illustrating the principal components of the present invention;

FIG. 2 is a general schematic diagram illustrating an alternative arrangement using the principal components of the present invention to cool the cerebrospinal fluid circulated over the brain and down the cerebrospinal axis over the spinal cord.

Like reference numerals refer to like parts throughout the drawings.

### DETAILED DESCRIPTION OF THE PREFERRED EMBODIMENT

In the central nervous system, cerebrospinal fluid generated in the ventricular system of the brain is slowly circulated down the cerebrospinal axis, over the spinal cord, and back up over the surface of the brain where it is then absorbed into the veins near the vertex of the brain. This process continues in a balanced manner.

Recent studies suggest that cooling the central nervous system under conditions of controlled hypothermia may dramatically increase the chance of survival in cases of severe trauma, while minimizing long-term damage to the central nervous system, and particularly the brain. There is a pressing medical need for a reliable method of cooling the central nervous system under controlled hypothermia. The ability to control cooling is critical to the protection of the central nervous system. Cooling the central nervous system under controlled hypothermia may be beneficial in the treatment of cancer as well as the protection of the central nervous system during cardiac arrest, stroke, spinal cord injury, and other conditions where the central nervous system may be at risk.

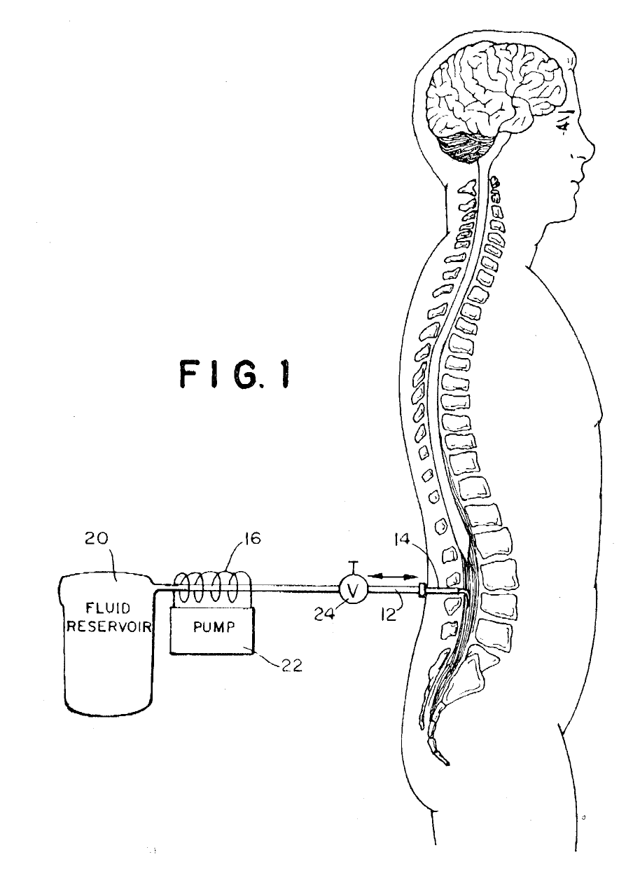

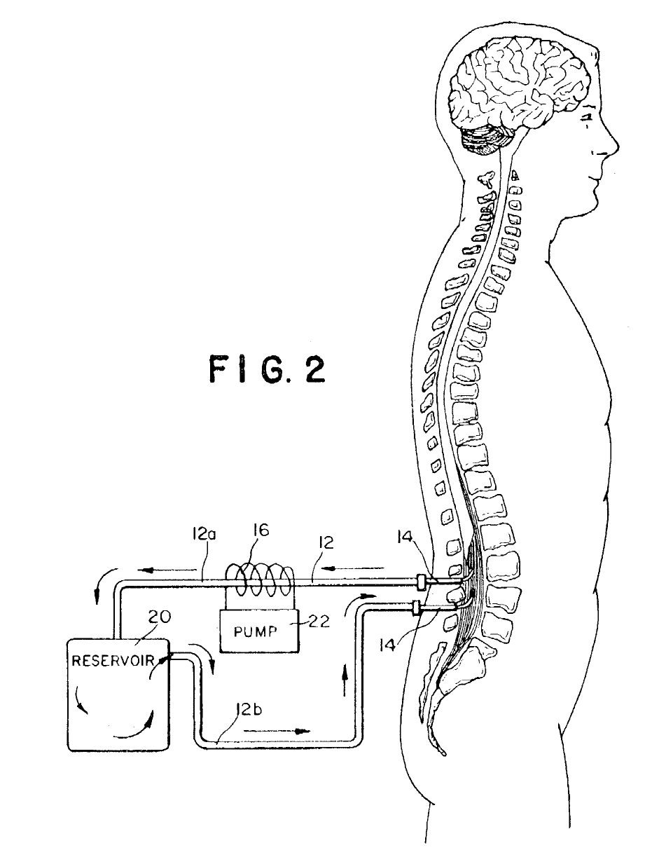

According to the present invention, a small flexible tube 12 is passed through a modified spinal needle 14 and into the subarachnoid space of the spinal canal. The flexible tube is passed through an external cooling coil 16, or other cooling device, or cooling solution, and is then returned to the subarachnoid space either through the same flexible tube, as seen in FIG. 1, or through a return tube, as seen in FIG. 2.

In a preferred embodiment, the cerebrospinal fluid is directed through the flexible tube 12 to a fluid reservoir 20. In either example (FIG. 1 or FIG. 2) the cerebrospinal fluid intermittently flows out of the spinal canal, through the flexible tube 12 and into the reservoir 20. The cerebrospinal fluid may be cooled either during travel through the flexible tube 12 or while in the reservoir 20. A pump device 22, such as a peristaltic pump, is used to control the direction and rate of flow through the flexible tube. Additionally, one or more valves 24 may be installed along the flexible tube 12 to limit direction of flow of the cerebrospinal fluid. For instance, as seen in FIG. 1, the spinal fluid may intermittently flow out of the spinal canal, through the flexible tube 12 and into the reservoir, while being cooled on the way to or while in the reservoir. Subsequently, valve 24 switches the flow to the reverse direction towards the spinal canal.

In the example in FIG. 2, cerebrospinal fluid is directed in a closed loop without interruption. Specifically, cerebral spinal fluid is directed through flexible tube 12a, through the cooling device, and into the reservoir, then returns through flexible tube 12b to the subarachnoid space where it is then circulated to the surface of the brain for absorption and cooling.

While the instant invention has been shown and described in accordance with preferred and practical embodiments thereof, it is recognized that departures from the instant disclosure are contemplated within the spirit and scope of the present invention.

What is claimed is:

1. A method of cooling the central nervous system comprising the steps of:
removing cerebrospinal fluid from the spinal canal;
cooling the cerebrospinal fluid; and
returning the cooled cerebrospinal fluid to the spinal canal.

2. The method as recited in claim 1 wherein the step of removing the cerebrospinal fluid from the spinal canal further comprises the step of:
removing the cerebrospinal fluid from the subarachnoid space of the spinal canal.

3. The method as recited in claim 2 further comprising the steps of:
inserting a spinal needle into the spinal canal; and
directing a flexible conduit into the subarachnoid space of the spinal canal.

4. The method as recited in claim 2 further comprising the step of:
circulating the cerebrospinal fluid through a flexible tube between said steps of removing the cerebrospinal fluid and returning the cerebrospinal fluid to the subarachnoid space of the spinal canal.

5. The method as recited in claim 4 wherein said step of cooling the cerebrospinal fluid further comprises the step of:
exposing the cerebrospinal fluid circulating through the flexible tube to a cooling coil for cooling the cerebrospinal fluid.

6. The method as recited in claim 4 further comprising the step of:
directing the cerebrospinal fluid to a reservoir between said steps of removing the cerebrospinal fluid and returning the cerebrospinal fluid to the subarachnoid space of the spinal canal.

7. The method as recited in claim 6 wherein said step of cooling the cerebrospinal fluid further comprises the step of:
cooling the cerebrospinal fluid in the reservoir prior to said step of returning the cerebrospinal fluid to the subarachnoid space of the spinal canal.

8. A method of cooling the central nervous system comprising the steps of:
removing cerebrospinal fluid from the subarachnoid space of the spinal canal;
cooling the removed cerebrospinal fluid; and
returning the cooled cerebrospinal fluid to the subarachnoid space of the spinal canal.

9. The method as recited in claim 8 further comprising the step of:
circulating the cerebrospinal fluid through a flexible tube between said steps of removing the cerebrospinal fluid and returning the cerebrospinal fluid to the subarachnoid space of the spinal canal.

10. The method as recited in claim 9 wherein said step of cooling the cerebrospinal fluid further comprises the step of:
exposing the cerebrospinal fluid circulating through the flexible tube to a cooling coil for cooling the cerebrospinal fluid.

11. The method as recited in claim 9 further comprising the step of:
directing the cerebrospinal fluid to a reservoir between the steps of removing the cerebrospinal fluid and returning the cerebrospinal fluid to the subarachnoid space of the spinal canal.

12. The method as recited in claim 11 wherein said step of cooling the cerebrospinal fluid further comprises the step of:
cooling the cerebrospinal fluid in the reservoir prior to said step of returning the cerebrospinal fluid to the subarachnoid space of the spinal canal.
